(19) United States
(12) Patent Application Publication
(10) Pub. No.: US 2022/0218360 A1
(43) Pub. Date: Jul. 14, 2022

(54) DEVICES AND METHODS FOR TREATING EDEMA

(71) Applicant: White Swell Medical Ltd, Kibbutz Shefayim (IL)

(72) Inventors: Yaacov Nitzan, Hertzelia (IL); Eamon Brady, Galway (IE)

(21) Appl. No.: 17/615,327

(22) PCT Filed: Jun. 2, 2020
(86) PCT No.: PCT/IB2020/000426

§ 371 Date: Nov. 30, 2021

Related U.S. Application Data
(60) Provisional application No. 62/857,060, filed on Jun. 4, 2019.

(51) Int. Cl. A61B 17/135; A61B 5/03; A61B 5/00

(52) U.S. Cl. CPC A61B 17/1355; A61B 2017/00876; A61B 5/6846; A61B 5/03

(57) ABSTRACT

Methods and devices that use a subcutaneous implant to express lymph from the lymphatic system to promote drainage, drive normal lymph flow, and avoid acute decompensated heart failure, without the requirement of an intravascular procedure or device. Embodiments include a treatment device with a subcutaneous implant dimensioned to be implanted and positioned at a thoracic duct of a subject and a controller operable to cause the implant to compress the thoracic duct to express lymph, optionally using an implantable pressure sensor, and in which the subcutaneous implant comprises a balloon or cuff that at least partially surrounds a thoracic duct, wherein, in response to a reading from the sensor indicative of inadequate lymph flow, the controller causes the implant to perform a series of transient compressions of the thoracic duct to express lymph.

### CROSS-REFERENCE TO RELATED APPLICATIONS

This applications claims priority to U.S. Provisional Application No. 62/857,060, filed on Jun. 4, 2019, the contents of which are incorporated by reference.

### TECHNICAL FIELD

The disclosure relates to devices and methods for the treatment of edema.

### BACKGROUND

Congestive heart failure occurs when the heart is unable to pump sufficiently to maintain blood flow to meet the body's needs. A person suffering heart failure may experience shortness of breath, exhaustion, and swollen limbs. Heart failure is a common and potentially fatal condition. In 2015 it affected about 40 million people globally and around 2% of adults overall. As many as 10% of people over the age of 65 are susceptible to heart failure.

In heart failure, the pressures in the heart ventricles and atria are excessively elevated. As a result, the heart works harder to eject blood, leading to a buildup of blood pressure, which may result in edema forming within interstitial compartments of the body. Edema refers to the abnormal accumulation of fluid in tissues of the body and results when elevated blood pressure prevents lymphatic fluid from draining from the interstitium. The additional work of the heart, with time, weakens and remodels the heart thus further reducing the ability of the heart to function properly. The fluid accumulation leads to dyspnea and acute decompensated heart failure (ADHF) hospitalization. Those conditions may result in severe health consequences including death.

### SUMMARY

The invention provides methods and devices that improve the flow of lymph in the lymphatic system to thereby drain lymphatic fluid and relieve abnormal accumulation of fluid from tissues within the body. Methods and devices of the disclosure can diminish the adverse effects of ADHF without the requirement an intravascular procedure or device and, in doing so, meet an unmet clinical need. Aspects of the invention are accomplished by externally applying pressure to a thoracic duct of a subject to increase pressure in the thoracic duct to a level that lymphatic fluid is propelled out of the thoracic duct and into venous circulation. In preferable embodiments of the disclosure, a subcutaneous implant positioned external to the thoracic duct is used to express lymph from the lymphatic system to promote drainage, drive normal lymph flow, and avoid ADHF. In other embodiments, a device that is completely external of the skin (i.e., outside of the body) is used to apply pressure to the thoracic duct.

Embodiments include treatment devices, and methods using such devices, that use a device external to the body or a subcutaneous implant dimensioned to be implanted and positioned at a thoracic duct of a subject and a controller operable to cause the device or implant to compress the thoracic duct to express lymph. Preferably, devices may include an implantable pressure sensor and the subcutaneous implant may include a balloon or cuff that can at least partially surrounds a thoracic duct. In response to a reading from the sensor indicative of inadequate lymph flow, the controller causes the implant (e.g., the balloon or cuff) to perform a series of transient compressions of the thoracic duct to express lymph.

Devices and methods of the invention are used to apply pressure to a thoracic duct of a subject to increase pressure in the thoracic duct to a level that lymphatic fluid is propelled out of the thoracic duct and into venous circulation. Preferably, the pressure is applied to a terminal lymphangion of (e.g., about the terminal 5 mm of) the thoracic duct, adjacent a junction of the thoracic duct and a vein. Devices and methods of the invention employ the insight that the last segment of the thoracic duct is defined between its two one-way valves: a downstream lymphatic valve at the outflow to the venous circulation and an upstream lymphatic valve defining a fluid entry point to that lymphangion. Typically each segment (lymphangion) is a few mm long. Compressing one or two or even three segments together will result in driving the lymph to the venous circulation as it cannot be driven backwards due to the one way valves. After draining the last few lymphangia the upstream lymphatics will drain into the segments that have been emptied as the pressure in those lymphangia will be low.

Methods and systems of the disclosure are also useful for locating the thoracic duct and the components thereof such as one or more terminal lymphangia thereof. Preferably, the duct or its components are located using ultrasound, lymphography, or computed tomography.

In certain aspects, the invention provides a method for treating a subject having a condition that involves reduced lymphatic flow. The method includes externally applying pressure to a thoracic duct of a subject to increase pressure in the thoracic duct to a level that lymphatic fluid is propelled out of the thoracic duct and into venous circulation, thereby increasing lymphatic flow and treating the condition. (Externally preferably means that no device is inserted into a lumen of the circulatory or lymphatic system.) The pressure is preferably applied to a distal five millimeters of the thoracic duct, and the pressure may be applied as a series of compressions to the thoracic duct. For example, the series of compressions may include about one to about twenty compressions are provided per minute.

The series of compressions may be provided by a device that is operably linked to a sensor. For example, the series of compressions may be provided on-demand by the device upon the device receiving a signal from the sensor of excess pressure in the thoracic duct or reduced lymphatic outflow from the thoracic duct.

In some magnetic embodiments, externally applying pressure is accomplished by bringing a first magnet into operable proximity to a second magnet, the second magnet being implanted within the subject and proximate the thoracic duct in a manner that the first and second magnetics repel each other, thereby causing the second magnet to apply pressure to the thoracic duct. The first magnet may be external to the subject. The dimensions of the magnet can be a few mm wide to 1-2 cm long. The provided first and second magnets may either or both be about a few mm wide to about one to two cm long or more. The first and second magnets may provide forces, e.g., strong enough repelling forces to press on the thoracic duct and collapse it when the first and second magnets are up to about 3 cm apart.

In certain inflation-based embodiments, externally applying pressure is accomplished by a device that comprises an inflatable member that is operably associated with, and external to, the thoracic duct. Inflation of the inflatable member applies pressure to the thoracic duct. Preferably, the inflatable member at least partially surrounds the thoracic duct. The inflatable member may be, for example, a balloon, and may fully surround the thoracic duct.

Externally applying pressure may be accomplished by applying pressure onto a neck of the subject at a location that corresponds to a location in which the compression will cause pressure to be applied to the thoracic duct. The condition may be a condition associated with high venous pressure greater than five mm Hg. The condition may be edema. The condition may be acute decompensated heart failure. Externally applying pressure may raise a pressure within a distal portion of the thoracic duct between about ten mm Hg and about thirty mm Hg.

Aspects of the disclosure provide a device for treating edema. The device includes a subcutaneous implant dimensioned to be implanted and positioned at a thoracic duct of a subject and a controller operable to cause the implant to compress the thoracic duct to thereby expel lymph from the thoracic duct and into a subclavian vein of the subject. The device may further include an (optional) implantable sensor operable to measure blood or lymphatic pressure within the subject. The controller may cause the implant to compress the thoracic duct in response to a reading from the sensor indicative of inadequate lymph flow.

In some embodiments, the implant comprises a subcutaneous magnet and the controller includes a second a magnet that is brought into proximity of the subject to cause the subcutaneous magnet to compress the thoracic duct. In certain embodiments, the subcutaneous implant comprises a balloon, cuff, or armature dimensioned to at least partially surround thoracic duct.

The device may include and use a sensor that measures pressure within the subject such that, in response to a reading from the sensor indicative of inadequate lymph flow, the controller causes the implant to compress the thoracic duct to thereby express lymph from the thoracic duct and into subclavian vein of the subject. The controller may cause the implant to perform a series of transient compressions of the thoracic duct. The series of compressions may have a substantially regular frequency, for example of at least about five compressions per minute.

In certain embodiments, the subcutaneous implant comprises armature dimensioned to at least partially surround thoracic duct.

The device may include an implantable sensor operable to measure blood or lymphatic pressure within the subject. The subcutaneous implant may operate as a balloon or cuff dimensioned to at least partially surround thoracic duct, wherein, in response to a reading from the sensor indicative of inadequate lymph flow, the controller causes the implant to perform a series of transient compressions of the thoracic duct. The series of compressions may have a substantially regular frequency of at least about five per minute. The balloon or cuff may be connected to a distal inflation lumen that terminates at a fitting, and the controller may include a proximal inflation lumen that comprises a complementary fitting, such that the implant can be implanted in the subject, and the controller can be subsequently connected via the fittings, to inflate the balloon or cuff to provide perform the series of transient compressions, to express lymph from the thoracic duct.

In certain aspects, the disclosure provides a device for treating a subject having a condition that involves an excess of fluid in the interstitial tissues of the subject. The device includes an energy element and a contacting element. The contacting element is configured to be placed in contact with the skin of the patient at a region of the thorax or neck of the subject. The device is operable to transduce an energy applied exterior of the patient into a pressure pulse in at least a part of the lymphatic system of a patient. The pressure pulse expresses lymph from a lymphangion and into a vein. Optionally, the energy element comprises an apparatus configured to deliver a pressure to the surface of the skin. The transducing of energy may include transducing a mechanical pulse across the skin and into a tissue in the region of the thoracic duct of the lymphatic system. The transducing of energy may include transducing a sonic pulse across the skin and into a tissue in the region of the thoracic duct of the lymphatic system. The transducing of energy may include delivering an electrical stimulus to effect a pulse in a tissue in the region of the thoracic duct of the lymphatic system.

In some embodiments, the contact element is configured to transduce energy to the thoracic duct while avoiding dampening structures, such as bones, in the body of the subject. The contact element may, responsive to the transduced energy, provide a series of transient pressure pulses (which series includes the pressure pulse). The series of transient pressure pulses are provided according to one or more pulse parameters. The pulse parameters may be designed to increase pressure in the thoracic duct. Preferably the increased pressure in the thoracic duct at least temporarily stimulates flow from the thoracic duct into the venous system. The pulse parameters may include one or more of pulse frequency, pulse length, pulse amplitude and pulse form.

### BRIEF DESCRIPTION OF THE DRAWINGS

FIG. 1 diagrams a method for treating reduced lymphatic flow.

FIG. 2 shows a patient with a subcutaneous implant device.

FIG. 3 is a close-up view of the device.

FIG. 4 shows a subcutaneous passive magnetic implant.

FIG. 5 shows a device with an inflatable implant.

FIG. 6 shows the device with a pressure sensor connected to the controller.

FIG. 7 shows a device for improving lymph flow.

### DETAILED DESCRIPTION

The invention provides devices and methods for treating edema. Devices and methods of the disclosure affect the lymphatic system, part of the circulatory system in conjunction with the arterial and venous systems. A primary function of the lymphatic system is to drain excessive interstitial fluid back into the venous system at two main locations: the thoracic duct and the lymphatic duct, which drain into the left and right subclavian veins, respectively.

Under normal circulatory conditions of the arterial and venous systems, the interstitial fluid volume balance is maintained and the lymph fluid is cleared back through the lymphatic system. Devices and methods of the disclosure are useful in pathological conditions such as edema and heart failure, in which the capillary hydrostatic pressure and the venous pulmonary pressure can become elevated and fluid flows excessively out of the blood vessels and into the interstitial and alveolar spaces. The pressure gradient between the initial lymphatics and at the outflow of the thoracic duct and the lymphatic duct is reduced and the lymphatic system cannot clear the additional fluid which accumulates in the air spaces of the lungs. This is a life threatening condition as gas exchange is impaired to the extent that it may lead to respiratory failure. Devices and methods of the disclosure are used to express lymph from the thoracic duct to relieve excess lymphatic pressure.

Devices and methods of the disclosure are used to apply pressure to a thoracic duct of a subject to increase pressure in the thoracic duct to a level that lymphatic fluid is propelled out of the thoracic duct and into venous circulation. Preferably, the pressure is applied to a terminal lymphangion of (e.g., about the terminal two to five mm of) the thoracic duct, adjacent a junction of the thoracic duct and a vein. Devices and methods of the invention employ the insight that the last segment of the thoracic duct is defined between its two one-way valves: a downstream lymphatic valve at the outflow to the venous circulation and an upstream lymphatic valve defining a fluid entry point to that lymphangion. Typically each segment (lymphangion) is a few mm long. Compressing one or two or even three segments together will result in driving the lymph to the venous circulation as it cannot be driven backwards due to the one way valves. After draining the last few lymphangia the upstream lymphatics will drain into the segments that have been emptied as the pressure in those lymphangia will be low.

Devices and methods may, in some embodiments, make use of an implantable pressure sensor in combination with an implant that can apply pressure to a lymphangion of the thoracic duct to express lymph. The implant may be operably responsive to a controller element (e.g., a magnetic device, a sonic device, or an inflation lumen), under control of a control system that uses the implantable pressure sensor to address symptomatic pressure anomalies in the lymphatic or general circulatory system. Importantly, the control system may be configured to use the implant to apply pressure to a thoracic duct in response to a pressure measured by the pressure sensor exceeding a predefined threshold that is indicative of edema or another such symptomatic pressure anomaly developing in the subject.

Lymph flow is influenced by factors such as outflow pressure in the venous angle, intrinsic pumping of the lymphatic lymphangion and its smooth muscle contraction force, and extrinsic forces of the surrounding tissues. Methods and devices herein are useful to empty the last lymphangion of the thoracic duct to initiate refilling and enhance lymph flow. A thoracic duct contracts normally only a few times per minute. The outflow pressure is normally at most about 20 mm Hg. Devices and methods of the disclosure are useful to treat patient populations in an acute phase and hospitalized. Devices and methods of the disclosure use a small subcutaneous implant and do not require an intravascular procedure, such as a trans-jugular introduction of a pump device. The subcutaneous implant may be used for ongoing or continual treatments of patients in a chronic phase of edema or heart failure.

Methods and devices of the disclosure may be used to increase pressure locally within a segment of a thoracic duct and generate extrinsic pumping to overcome venous pressure, to thereby express lymph from the thoracic duct. To restore lymph draining over a period of time, one may use methods and devices of the disclosure to locally apply gentle cyclic external force (to thoracic duct at a final or terminal lymphangion) to increase the pressure inside it to overcome elevated venous pressure and empty duct. A lymphangion is the functional unit of a lymph vessel that lies between two semilunar (half-moon shaped) valves. Those valves essentially restrict fluid flow to a single direction. In the terminal lymphangion, the valves allow fluid to only flow out of the thoracic duct and into a vein of the venous angle, such as the subclavian vein.

In certain embodiments, a small surgical procedure is used to place an implant (e.g., subcutaneously) adjacent to or proximal to a lymphangion. The implant is operated to apply a pressure pulse to squeeze the lymphatic duct, thereby pushing fluid out of the duct, via the valves, into venous circulation. The pressure pulse is transient and when the pressure is relieved, that lymphangion will have a lower pressure than “upstream” parts of the lymphatic system. Due to the pressure differential, lymph will flow from the upstream parts of the lymphatic system into that lymphangion. A subsequent pressure pulse causes another cycle of lymph expression and flow. By delivering transient pressure pulses in a cycle, or periodically, drainage of lymph is promoted, which aids to prevent ADHF episodes. The devices and methods may be used during ADHF episodes to aid the patient or between episodes.

Methods and devices of the disclosure may use any suitable mechanism or technique to apply pressure (e.g., cyclic pressure) onto the last 5 mm of the thoracic duct.

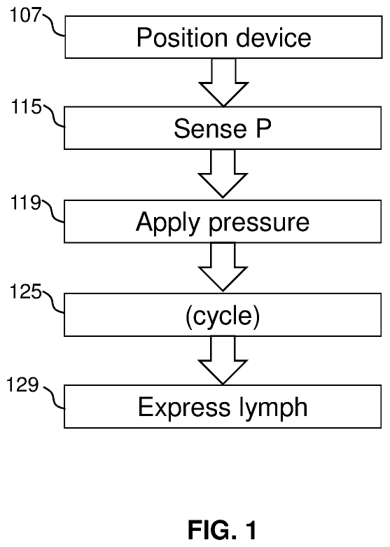

FIG. 1 diagrams a method for treating a subject having a condition that involves reduced lymphatic flow. The method includes externally applying 119 pressure to a thoracic duct of a subject to increase pressure in the thoracic duct to a level that lymphatic fluid is expressed 129 out of the thoracic duct and into venous circulation, thereby increasing lymphatic flow and treating the condition. The method optionally includes positioning 107 a subcutaneous implant in the subject. For example, the implant may be positioned one time, via a surgical procedure, to allow for subsequent episodes of applying 119 pressure and expressing 129 lymph. The method may not include placing 107 the device when, for example, the device has positioned 107 previously and the patient is known to have the implant.

The subcutaneous implant is preferably positioned 107 at, adjacent to, around, substantially surrounding, proximal to, or in contact with at least a segment of a thoracic duct. Operation of the device can then apply pressure 119 to the duct. Preferably, the pressure is applied to a distal five millimeters of the thoracic duct.

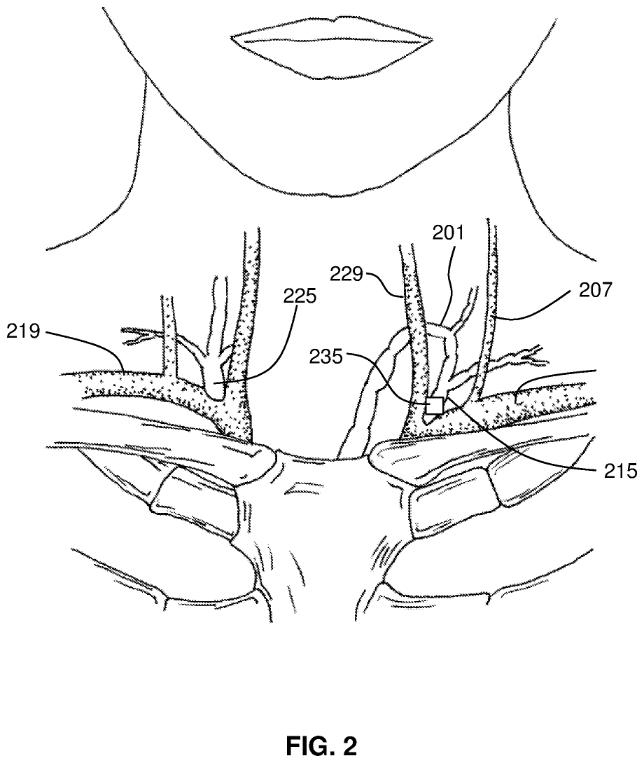

FIG. 2 shows a patient with a subcutaneous implant 235 positioned at a terminal 5 mm of a thoracic duct 201. As shown, the implant 235 is adjacent a terminal lymphangion 215 of the duct. It can be seen that the thoracic duct 201 terminates at the subclavian vein, which drains the internal jugular vein 229 and the external jugular vein 207. For reference, the right lymphatic duct 225 is shown, draining into the subclavian vein 219.

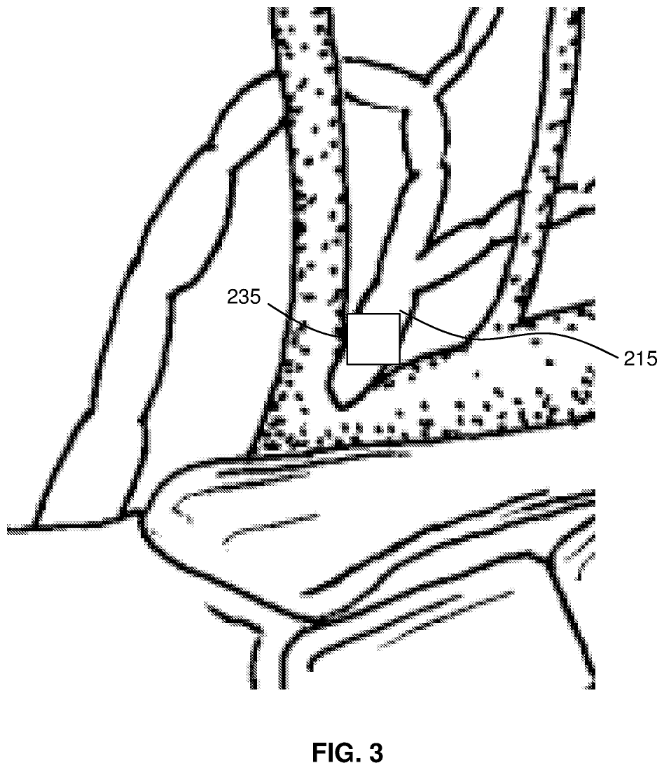

FIG. 3 is a close-up view of the device 235 positioned at a terminal lymphangion 215 of the thoracic duct. The implant 235 is operable to apply 119 pressure to the duct 201. Preferably, the pressure is applied as a series of compressions to the thoracic duct. Optionally, about one to about twenty compressions are provided per minute.

Any suitable device or mechanism may be used for the implant 235. For example, the device may include a balloon that is inflated and deflated. In other embodiments, pressure is applied by a completely external mechanical device that applies compression through a local point on the neck.

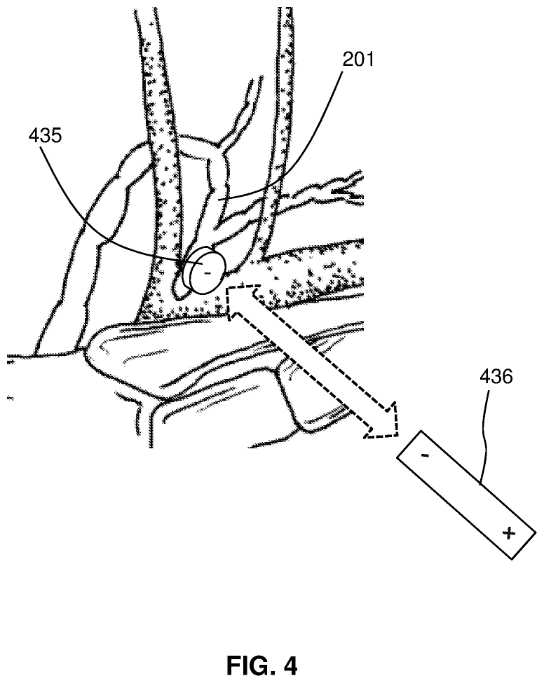

FIG. 4 illustrates an embodiment in which a subcutaneous passive implant 435 (e.g., a magnet) is externally activated by a magnetic controller 436. The controller 436 operates as an external magnet. The implant 435 is pushed away from the external magnet and compresses the duct (e.g., cyclically). The external magnet may be, for example, an electromagnet (e.g., in a handheld wand or mounted on a rig) and under changing current, the electromagnet of the controller 436 may exhibit changing (e.g., alternating) magnetic poles. To compress thoracic duct 201, the controller may be operated to change to present a negative pole such that the external magnet will push the internal magnet implant 435 to compress the duct 201.

Using the implant 435 and controller 436, externally applying pressure is accomplished by bringing a first magnet into operable proximity to a second magnet, the second magnet being implanted within the subject and proximate the thoracic duct in a manner that the first and second magnetics repel each other, thereby causing the second magnet to apply pressure to the thoracic duct. Preferably, the first magnet is external to the subject. The dimensions of either magnet can be a few mm wide to 1-2 cm long. The provided first and second magnets may either or both be about a few mm wide to about one to two cm long or more. The first and second magnets may provide forces, e.g., strong enough repelling forces to press on the thoracic duct and collapse it when the first and second magnets are up to about 3 cm apart.

Devices and methods of the disclosure may be used to create a pressure rise of between about 10 mm Hg and about 30 mm Hg in the last few millimeters of the thoracic duct. Devices and methods of the disclosure may be used to create between about 5 and about 15 compressions per minute. It is expected that the proposed cyclic pulses of pressure may increase lymph flow through the thoracic duct by at least about five milliliters per minute. The lymph will propel forward since upstream there are one way valves in the lymphatic system that prevent retrograde flow.

In some embodiments, the series of compressions are provided by a device that comprises a sensor and the series of compressions are provided on-demand by the device upon the device receiving a signal from the sensor of excess pressure in the thoracic duct or reduced lymphatic outflow from the thoracic duct.

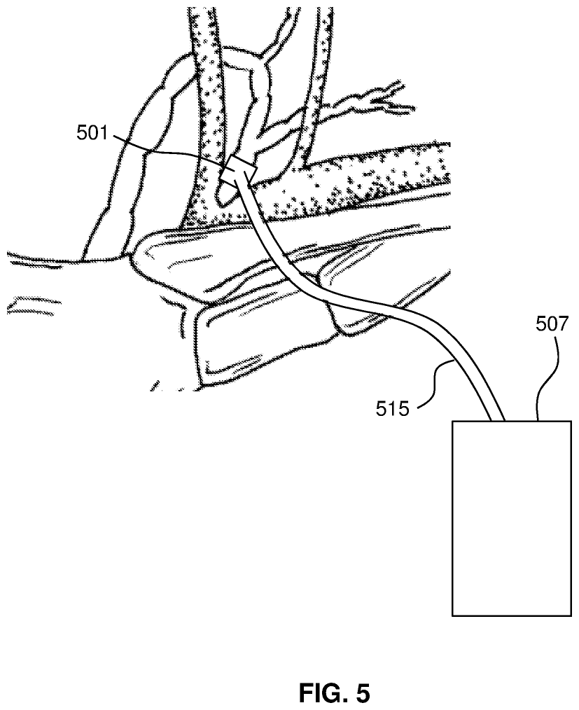

FIG. 5 shows a device that includes a subcutaneous implant 501 dimensioned to be implanted and positioned at a thoracic duct of a subject and a controller 507 operable to cause the implant 501 to compress the thoracic duct to express lymph. The implant 501 may include a cuff, or a balloon, or armature (e.g., articulated jaws under motorized control to provide a clamping force) dimensioned such that the device 501 may be positioned to at least partially surround the thoracic duct. Where the device 501 is motorized, the controller 501 can provide the logic and signals to drive the motorized compressions. Where the device 501 is inflatable, it may be connected to the controller 507 via an inflation lumen 515, such the controller may express lymph from the duct by inflating the cuff or balloon of implant 501 to squeeze the duct. The squeezing, or pressure pulse, may be applied periodically or cyclically, or under control from reading a pressure sensor or other such input. Embodiments of the disclosure do not require feedback of a pressure sensor to initiate the cyclic compressions. For example, the device may be operated upon the initiation or control input of a physician or technician. The device may be optionally linked to an optional pressure sensor. However, in one preferred embodiment, the device does not need feedback of any pressure sensor to initiate cyclic compressions. Upon being activated by a user (e.g., physician), the implant 501 initiates cyclic compression of a lymphangion such as the terminal lymphangion of the thoracic duct. In other embodiments, a pressure sensor is used.

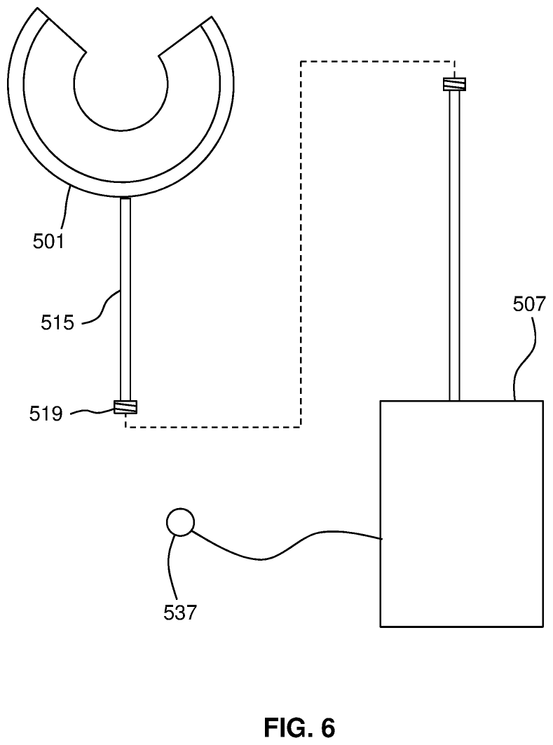

FIG. 6 shows the device with a pressure sensor 537 connected to the controller. Here, the device uses one or more of the optional implantable pressure sensor 537 to determine pressure at one or more appropriate locations within the patient's lymphatic or circulatory system. In the depicted embodiment, the subcutaneous implant 501 includes a balloon or cuff that at least partially surrounds a thoracic duct. For example, the implant 501 may include a rigid or semi-rigid “C”-shaped brace with an inflatable balloon or cuff disposed along and around an inward-facing surface of the C-shaped brace. An inflation lumen 515 passes through the brace and connects to the balloon, allowing it to be inflated. The brace restricts the inflating balloon to occupy a greater volume of space within the inside area of the C-shaped brace. Thus, when the C-shaped brace is disposed about duct (e.g., a thoracic duct), inflation of the balloon squeezes the duct, applying a transient pressure pulse to the duct.

As shown, the balloon or cuff implant 501 is connected to a distal inflation lumen 515 that terminates at a fitting 519. The controller 507 comprises a proximal inflation lumen that comprises a complementary fitting. By means of the fittings that may be coupled to each other, the implant can be implanted in the subject and the controller can be subsequently attached via the fitting and complementary fitting, to inflate the balloon or cuff to provide perform the series of transient compressions, to express lymph from the thoracic duct.

In some embodiments, the device 501 is used such that in response to a reading from the sensor 537 indicative of inadequate lymph flow, the controller 507 causes the implant 501 to perform a series of transient compressions of the thoracic duct to express lymph.

Thus, externally applying pressure may be accomplished by a device 501 that comprises an inflatable member that is operably associated with, and external to, the thoracic duct, wherein inflation of the inflatable member applies pressure to the thoracic duct. Here, the inflatable member (of implant 501) at least partially surrounds the thoracic duct. In other embodiments, the inflatable member fully surrounds the thoracic duct.

The subcutaneous implant 501 (e.g., a balloon, cuff, or armature dimensioned to at least partially surround thoracic duct) is dimensioned to be implanted and positioned at a thoracic duct of a subject. The implant 501 may be placed under radiographic guidance, and the implant procedure may be on a different day, or at a different location, the use of the device. The implant 501 is operated via the controller 507 to cause the implant to compress the thoracic duct to express lymph. One or more of the sensor 537 may also be implanted. In response to a reading from the sensor 537 indicative of inadequate lymph flow, the controller 507 causes the implant to perform a series of transient compressions of the thoracic duct to express lymph. Preferably, the controller 507 causes the implant 501 to perform a series of transient compressions of the thoracic duct, e.g., at a substantially regular frequency (e.g., at least about 5 to 15 compressions per minute).

In general and among the embodiments and aspects of the disclosure it can be seen that embodiments of the disclosure provide a device for treating a subject having a condition that involves an excess of fluid in the interstitial tissues of the subject, in which the device includes an energy element and a contacting element, the contacting element configured to be placed in contact with the skin of the patient at a region of the thorax or neck of the subject, the device further configured to transduce an energy applied exterior of the patient into a pressure pulse in at least a part of the lymphatic system of a patient.

The pressure pulse expresses lymph from a lymphangion and into a vein. The energy element may include an apparatus configured to deliver a pressure to the surface of the skin. The transducing of energy may include transducing a mechanical pulse across the skin and into a tissue in the region of the thoracic duct of the lymphatic system. The transducing of energy may include transducing a sonic pulse across the skin and into a tissue in the region of the thoracic duct of the lymphatic system. The transducing of energy may include delivering an electrical stimulus to effect a pulse in a tissue in the region of the thoracic duct of the lymphatic system.

The contact element is configured to transduce energy to the thoracic duct while avoiding dampening structures, such as bones, in the body of the subject. Preferably, the contact element, responsive to the transduced energy, provides a series of transient pressure pulses, wherein the series of transient pressure pulses includes the pressure pulse. The series of transient pressure pulses may be provided according to one or more pulse parameters. The pulse parameters may be optimized to increase pressure in the thoracic duct. The increased pressure in the thoracic duct at least temporarily stimulates flow from the thoracic duct into the venous system. The pulse parameters may include one or more of pulse frequency, pulse length, pulse amplitude and pulse form. In some embodiments, the condition is a condition associated with high venous pressure greater than five mm Hg. The condition may be edema. The condition may be acute decompensated heart failure. Preferably externally applying pressure raises a pressure within a distal portion of the thoracic duct between about ten mm Hg and about thirty mm Hg.

Devices and methods of the disclosure include fully-external embodiments that do not require any intravascular or subcutaneous device or implant.

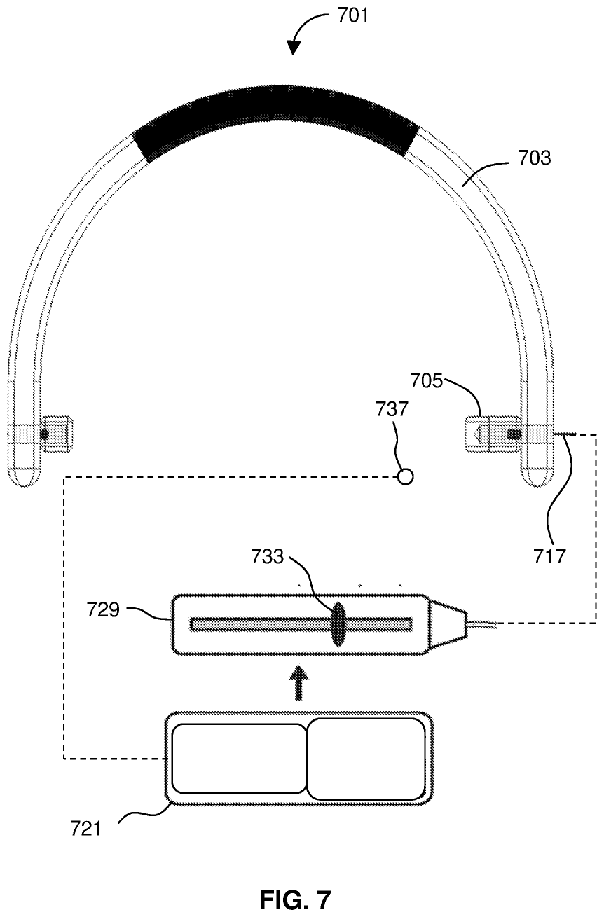

FIG. 7 shows a device 701 for treating edema. The device 701 includes a wearable member 703 dimensioned to be placed around a neck or over a shoulder of a subject. The wearable member includes a protrusion 705 located on the wearable member such that the wearable member 703 can be placed on or around a part of a patient's body (e.g., around a neck or over a shoulder) with the protrusion sitting on the patient's skin near a terminal segment of a thoracic duct. The protrusion is preferably extendable or expandable. For example, the protrusion may be threaded onto a motorized rotating post such that rotating the threaded post drives the protrusion 705 into the patient's skin, resulting in compression of the terminal few millimeters of the thoracic duct, which compression expresses lymph from the duct into the circulatory system.

In some embodiments, the protrusion 705 is an inflatable element or balloon connected via an inflation lumen 717 to a pump 729. The wearable member 703 may include a plastic material allowing the device 701 to be stretched and placed around the patient's neck and/or shoulder. By virtue of its plasticity, the wearable member 703 may grip the neck and/or shoulder and hold the position of the protrusion adjacent the terminal segment, or lymphangion, of the thoracic duct. Preferably, the device 701 includes a controller 721 operable to cause the protrusion 705 to compress the thoracic duct to thereby expel lymph from the thoracic duct and into a subclavian vein of the subject. The device 701 may also include an implantable sensor 737 operable to measure blood or lymphatic pressure within the subject, in which embodiments, the controller 721 may include software logic that drives the expansion or extension of the protrusion to provide a series of compressions to the terminal lymphangion. For example, where the protrusion includes an inflatable balloon, the controller 721 can issue instructions to a pump 729 to inflate the balloon repeatedly or at least a few times. Thus the controller 721 causes the protrusion 705 to compress the thoracic duct in response to a reading from the sensor 737 indicative of inadequate lymph flow. Thus, for certain embodiments of device 701, in response to a reading from the sensor 737 indicative of inadequate lymph flow, the controller 721 causes the protrusion 705 to compress the thoracic duct (e.g., by inflating the balloon via pump 729) to thereby express lymph from the thoracic duct and into subclavian vein of the subject. The controller 721 may cause the protrusion 705 to perform a series of transient compressions of the thoracic duct. The series of compressions have a substantially regular frequency, e.g., of at least about five compressions per minute.

With the device 701, the disclosure provides an extracorporeal method for treating a subject having a condition that involves reduced lymphatic flow. The extracorporeal method includes externally applying pressure to a thoracic duct of a subject to increase pressure in the thoracic duct to a level that lymphatic fluid is expressed out of the thoracic duct and into venous circulation, thereby increasing lymphatic flow and treating the condition. The extracorporeal method is extracorporeal in that no intravascular or subcutaneous devices is used to apply the pressure to the thoracic duct. Preferably, in the extracorporeal method the pressure is applied to a distal five millimeters of the thoracic duct. The pressure may be applied by a series of compressions to the thoracic duct. Optionally, about one to about twenty compressions are provided per minute. In some embodiments of the extracorporeal method, the series of compressions are provided by a device 701 that comprises a sensor 737 and the series of compressions are provided on-demand by the device upon a controller 721 of the device receiving a signal from the sensor 737 of excess pressure in the thoracic duct or reduced lymphatic outflow from the thoracic duct. In the extracorporeal method, the device 701 may include a wearable member 703 dimensioned to be placed around a neck or over a shoulder of a subject. The wearable member may include a protrusion 705 located on the wearable member such that the wearable member 703 can be placed on or around a part of a patient's body (e.g., around a neck or over a shoulder) with the protrusion sitting on the patient's skin near a terminal segment of a thoracic duct. The protrusion is preferably extendable or expandable. For example, the protrusion may be threaded onto a motorized rotating post such that rotating the threaded post drives the protrusion 705 into the patient's skin, resulting in compression of the terminal few millimeters of the thoracic duct, which compression expresses lymph from the duct into the circulatory system.

In preferred embodiments of the extracorporeal method, the protrusion 705 uses an inflatable member, such as a balloon. Externally applying pressure may be accomplished by applying pressure, via the protrusion, onto the subject at a location that corresponds to a location in which the compression will cause pressure to be applied to the thoracic duct. The condition may be a condition associated with high venous pressure greater than about twenty mm Hg. The condition may be edema or acute decompensated heart failure. Externally applying pressure preferably raises a pressure within a distal portion of the thoracic duct between about ten and about thirty mm Hg by virtue of the device 701.

The device 701 is useful to improve the flow of lymph in the lymphatic system to thereby drain lymphatic fluid and relieve abnormal accumulation of fluid from tissues within the body. In the depicted embodiment, the device 701 is designed to be completely external of the skin (i.e., outside of the body) is used to apply pressure to the thoracic duct. Methods using the device 701 can diminish the adverse effects of ADHF without the requirement of an intravascular procedure or device and, in doing so, meet an unmet clinical need. The device 701 is useful for externally applying pressure to a thoracic duct of a subject to increase pressure in the thoracic duct to a level that lymphatic fluid is expressed from the thoracic duct and into venous circulation.

The device 701 includes a pump 729 such as a programmable pump that can control inflation of, and time-varying patterns of inflation of, the inflatable pad 705. A volume of the inflatable pad 705 may be controlled by a pump 729 (e.g., an oscillating pump) in a closed-air system to allow rapid bidirectional volume transfer. In some embodiments, motion of the pump piston 733 is controlled by a control module 721 (e.g., running on a computer system). Optionally, based on input from a pressure sensor 737, the control module 721 synchronizes the forward and backward motion of the piston 733 to apply a series of transient compressions to the segment of a thoracic duct.

Any suitable pump may be used. For example, in some embodiments, the pump has a 24 mm diameter, 50 mm stroke length air-filled antifriction cylinder, e.g., as available from Airpot Corp (Norwalk, Conn.), with a piston driven by a stepper motor using a ball screw linear actuator (Model EZC6-05, Oriental Motors Co., Ltd), via a ball-joint interconnection. The stepper motor is, in turn, controlled and driven by a dedicated motor controller/driver, e.g., as available from Oriental Motor U.S.A. Corp. (Torrance, Calif.).

The disclosure generally relates to devices and methods for the treatment of edema. Details may be found in Chikly, 2005, Manual techniques addressing the lymphatic system: origins and development, JAOA 105(10):457-464; Ratnayake, 2018, The anatomy and physiology of the terminal thoracic duct and ostial valve in health and disease: potential implications for intervention, J Anat 233:1-14; and U.S. Pub. 2016/0166463 A1, the contents of which are all incorporated by reference.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification, and guidance that can be adapted to the practice of this invention in its various embodiments and equivalents thereof.

1. A method for treating a subject having a condition that involves reduced lymphatic flow, the method comprising:
externally applying pressure to a thoracic duct of a subject to increase pressure in the thoracic duct to a level that lymphatic fluid is propelled out of the thoracic duct and into venous circulation, thereby increasing lymphatic flow and treating the condition.

2. The method of claim 1, wherein the pressure is applied to a distal five millimeters of the thoracic duct.

3. The method of claim 1, wherein the pressure is applied by a series of compressions to the thoracic duct.

4. The method of claim 3, wherein about one to about twenty compressions are provided per minute.

5. The method of claim 3, wherein the series of compressions are provided by a device that comprises a sensor and the series of compressions are provided on-demand by the device upon the device receiving a signal from the sensor of excess pressure in the thoracic duct or reduced lymphatic outflow from the thoracic duct.

6. The method of claim 1, wherein externally applying pressure is accomplished by bringing a first magnet into operable proximity to a second magnet, the second magnet being implanted within the subject and proximate the thoracic duct in a manner that the first and second magnetics repel each other, thereby causing the second magnet to apply pressure to the thoracic duct.

7. The method of claim 6, wherein the first magnet is external to the subject.

8. The method of claim 1, wherein externally applying pressure is accomplished by a device that comprises an inflatable member that is operably associated with, and external to, the thoracic duct, wherein inflation of the inflatable member applies pressure to the thoracic duct.

9. The method of claim 8, wherein the inflatable member at least partially surrounds the thoracic duct.

10. The method of claim 8, wherein the inflatable member fully surrounds the thoracic duct.

11-16. (canceled)

17. A device for treating edema, the device comprising:
a subcutaneous implant dimensioned to be implanted and positioned at a thoracic duct of a subject; and
a controller operable to cause the implant to compress the thoracic duct to thereby expel lymph from the thoracic duct and into a subclavian vein of the subject.

18-19. (canceled)

20. The device of claim 17, wherein the implant comprises a subcutaneous magnet and the controller includes a second a magnet that is brought into proximity of the subject to cause the subcutaneous magnet to compress the thoracic duct.

21. The device of claim 17, wherein the subcutaneous implant comprises a balloon, cuff, or armature dimensioned to at least partially surround thoracic duct.

22. The device of claim 21, further comprising a sensor that measures pressure within the subject.

23. The device of claim 22, wherein, in response to a reading from the sensor indicative of inadequate lymph flow, the controller causes the implant to compress the thoracic duct to thereby express lymph from the thoracic duct and into subclavian vein of the subject.

24. The device of claim 17, wherein the controller causes the implant to perform a series of transient compressions of the thoracic duct.

25. The device of claim 24, wherein the series of compressions has a substantially regular frequency that includes at least about 5 compressions per minute.

26-27. (canceled)

28. The device of claim 17, further comprising an implantable sensor operable to measure blood or lymphatic pressure within the subject, wherein the subcutaneous implant comprises a balloon or cuff dimensioned to at least partially surround thoracic duct, wherein, in response to a reading from the sensor indicative of inadequate lymph flow, the controller causes the implant to perform a series of transient compressions of the thoracic duct.

29. The device of claim 28, wherein the series of compressions has a substantially regular frequency of at least about 5 per minute.

30. The device of claim 28, wherein the balloon or cuff is connected to a distal inflation lumen that terminates at a fitting, wherein the controller comprises a proximal inflation lumen that comprises a complementary fitting, and wherein the implant can be implanted in the subject, and the controller can be subsequently attached via the fitting and complementary fitting, to inflate the balloon or cuff to provide perform the series of transient compressions, to express lymph from the thoracic duct.

31-42. (canceled)
